(19) United States
(12) Patent Application Publication
(10) Pub. No.: US 2024/0249814 A1
(43) Pub. Date: Jul. 25, 2024

(54) Method And Apparatus For Audio Playback

(71) Applicant: Anhui Huami Technology Co., Ltd., Hefei (CN)

(72) Inventors: Wang Huang, Hefei (CN); Kongqiao Wang, Hefei (CN); Jinian Yan, Hefei (CN); Guokang Zhu, Hefei (CN); Zi Meng, Hefei (CN); Cong Zhang, Hefei (CN); Yi Yu, Hefei (CN)

(21) Appl. No.: 18/603,913

(22) Filed: Mar. 13, 2024

Related U.S. Application Data
(63) Continuation of application No. PCT/CN2022/114355, filed on Aug. 23, 2022.

(30) Foreign Application Priority Data
Jan. 6, 2022 (CN) 202210008594.5

(51) Int. Cl. G16H 20/30; A61B 5/00; A61B 5/0205; A61B 5/021; A61B 5/024; A61B 5/08; A61B 5/11; A61B 5/16; G16H 10/60; G16H 20/70

(52) U.S. Cl. CPC G16H 20/30; A61B 5/0205; A61B 5/681; A61B 5/7405; G16H 10/60; G16H 20/70; A61B 5/021; A61B 5/02405; A61B 5/0816; A61B 5/1118; A61B 5/165

(57) ABSTRACT

Provided a method, an apparatus, a wearable device and a storage medium for audio playback, and relates to the fields of machine learning, deep learning, intelligent wearable, etc. The method includes: measured vital sign data of a user within a first time range is obtained; a vital sign prediction is performed based on the measured vital sign data within the first time range, to obtain predicted vital sign data of the user within a second time range after the first time range; one or more target audio parameters are determined based on the predicted vital sign data and user attribute information of the user; and matched target audio data is obtained based on the one or more target audio parameters.

### CROSS-REFERENCE TO RELATED APPLICATION(S)

The present application is a continuation of PCT/CN2022/114355, filed Aug. 23, 2022, which claims priority and benefit of Chinese Patent Application No. 202210008594.5, filed Jan. 6, 2022, the entire contents of both of which are incorporated herein by reference.

### TECHNICAL FIELD

The present disclosure relates to the field of computer technologies, in particular to the field of machine learning, deep learning, smart wearables, etc., in particular to a method and an apparatus for audio playback, a wearable device and a storage medium.

### BACKGROUND

With the development and change of modern society, the pace of work and life is accelerating, which also puts higher requirements for people's spiritual and physical adaptability. More and more people are faced with sub-health problems such as insomnia, obesity, depression, and so on, which gives birth to a variety of ways to regulate and improve sleep and/or health. Audio playback is a method to adjust and improve users' health based on vital sign data.

However, in actual playback, audio often does not match the users' signs, resulting in poor improvement of the users' health.

### SUMMARY

In order to achieve the above purpose, the first aspect of the present disclosure provides a method for audio playback, including:
obtaining measured vital sign data of a user within a first time range;
performing a vital sign prediction based on the measured vital sign data within the first time range, to obtain predicted vital sign data of the user within a second time range after the first time range;
determining one or more target audio parameters based on the predicted vital sign data and user attribute information of the user; and obtaining matched target audio data based on the one or more target audio parameters.

In some implementations, obtaining measured vital sign data of the user within the first time range includes:
obtaining the measured vital sign data of the user within the first time range based on user scenario information, in which the user scenario information is used for indicating health requirements of the user.

In some implementations, obtaining measured vital sign data of the user within the first time range based on the user scenario information includes:
in response to the user scenario information indicating a sleep aid scenario or a focus scenario, obtaining heart rate data and activity data of the user within the first time range; or
in response to the user scenario information indicating a weight loss scenario, obtaining at least one of activity data, motion data, heart rate data, or positioning data of the user within the first time range; or
in response to the user scenario information indicating an emotion regulation scenario, obtaining heart rate variability of the user within the first time range.

In some implementations, performing the vital sign prediction based on the measured vital sign data within the first time range, to obtain the predicted vital sign data of the user within the second time range after the first time range includes:
performing feature extraction processing on the measured vital sign data within the first time range, to obtain first feature data;
performing fusing processing on the first feature data and the user attribute information, to obtain second feature data; and
performing the vital sign prediction based on the second feature data, to obtain the predicted vital sign data within the second time range.

In some implementations, determining one or more target audio parameters based on the predicted vital sign data and user attribute information of the user includes:
determining a target physiological parameter of the user; and
determining the one or more target audio parameters based on the predicted vital sign data, the target physiological parameter and user attribute information.

In some implementations, determining the target physiological parameter of the user includes:
determining the target physiological parameter of the user based on at least one of user scenario information or historical vital sign data of the user.

In some implementations, determining the target physiological parameter of the user includes:
in response to the user scenario information indicating a sleep aid scenario or a focus scenario, determining target activeness of the user; or
in response to the user scenario information indicating a weight loss scenario, determining at least one of target activeness, target activity amount and target heart rate of the user; or
in response to the user scenario information indicating an emotion regulation scenario, determining heart rate variability of the user.

In some implementations, the target physiological parameter includes activeness;
determining the target physiological parameter of the user includes:
obtaining historical heart rate data and historical activity data of the user within a third time range corresponding to the first time range;
determining the historical activeness of the user based on the historical heart rate data and the historical activity data of the user; and
determining target activeness of the user based on the historical activeness of the user, the target activeness is less than the historical activeness.

In some implementations, determining one or more target audio parameters based on the predicted vital sign data and user attribute information of the user includes:
inputting the predicted vital sign data, the target physiological parameter and the user attribute information into a regression model, to obtain the one or more target audio parameters outputted by the regression model, the one or more target audio parameters include target audio loudness and target rhythm.

In some implementations, the one or more target audio parameters include a target audio modulation parameter;
obtaining matched target audio data based on the one or more target audio parameters includes:
sending the target audio modulation parameter to a server, so as to enable the server to query target audio data matching the target audio modulation parameter; and
obtaining the target audio data sent by the server.

According to the method for audio playback in the implementation of the present disclosure, the target physiological parameter is determined based on user scenario information; one or more target audio parameters are determined based on the target physiological parameter of the user; and matched target audio data is determined based on the one or more target audio parameters. The user scenario information is used for indicating health requirements of the user. In this way, the one or more target audio parameters are determined which help the user to achieve the target physiological parameter by a matching model of target physiological parameter and one or more target audio parameters based on the health requirements of the user. According to this active modulation method, a target audio is effectively determined based on health requirements of the user, so as to better improve health status of the user.

In order to achieve the above purpose, an apparatus for audio playback is provided in a third aspect of the present disclosure, which includes:
a collecting module, configured to obtain measured vital sign data of a user within a first time range;
a predicting module, configured to perform a vital sign prediction based on the measured vital sign data within the first time range, to obtain predicted vital sign data of the user within a second time range after the first time range;
a first determining module, configured to determine one or more target audio parameters based on the predicted vital sign data and user attribute information of the user; and
an acquiring module, configured to obtain matched target audio data based on the one or more target audio parameters.

In order to achieve the above purpose, a wearable device is provided in a fourth aspect of the disclosure, and includes: at least one processor; and a memory communicatively coupled to the at least one processor; the memory is stored with instructions executable by the at least one processor, the instructions are performed by the at least one processor, the at least one processor is caused to perform the method for audio playback as described in the first aspect of the disclosure or the method for audio playback as described in the second aspect of the disclosure.

In order to achieve the above purpose, a computer readable storage medium stored with computer instructions is provided in a fifth aspect of the disclosure. The computer instructions are configured to execute the method for audio playback as described in the first aspect and the method for audio playback as described in the second aspect by the computer.

In order to achieve the above purpose, a computer program product is provided in a sixth aspect of the disclosure, and includes a computer program, the computer program executes the method for audio playback as described in the first aspect and the method for audio playback as described in the second aspect when executed by a processor.

Additional aspects and advantages of the present disclosure will be set forth in part in the following description, and in part will become obvious from the following description, or may be learned by practice of the disclosure.

### BRIEF DESCRIPTION OF THE DRAWINGS

FIG. 1 is a flowchart illustrating a method for audio playback provided in an implementation of the present disclosure;

FIG. 2 is a schematic diagram of a principle of audio playback provided in the implementations of the present disclosure;

FIG. 3 is a flowchart of a method for audio playback provided in another implementation of the present disclosure;

FIG. 4 is a schematic diagram of a prediction model provided in the implementation of the present disclosure;

FIG. 5 is a schematic diagram of a regression model provided in the implementation of the present disclosure;

FIG. 6 is a flowchart of a method for audio playback provided in another implementation of the present disclosure;

FIG. 7 is a structural diagram of an apparatus for audio playback provided in the implementation of the present disclosure;

FIG. 8 is a structural diagram of an apparatus for audio playback provided in another implementation of the present disclosure; and

FIG. 9 is a structural diagram of a wearable device provided in the implementation of the present disclosure.

### DETAILED DESCRIPTION

Implementations of the present disclosure are described in detail below, and examples of implementations are illustrated in the accompanying drawings, in which the same or similar labels represent the same or similar elements or elements with the same or similar functions. The implementations described below with reference to the drawings are examples, which are intended to be configured to explain the present disclosure and are not to be construed as a limitation of the present disclosure.

At present, an audio is modulated and played based on a latest measured vital sign state of a user. However, if the audio is modulated and played based only on the latest measured vital sign state of the user, when the audio is being played, the audio lags behind vital sign of the user, which makes the audio not match the vital sign of the user, and makes it difficult to meet personalized modulation requirements of the user.

Therefore, in view of the above problems, implementations according to the present disclosure proposes a method, an apparatus for audio playback, a wearable device and a storage medium to determine one or more target audio parameters based on predicted vital sign data, a target physiological parameter and user attribute information, which solves the problem that the actual audio played does not match the requirements of the user and the adjustment effect is poor.

A method and an apparatus for audio playback, a wearable device and a storage medium are described referring to figures in implementations of the present disclosure.

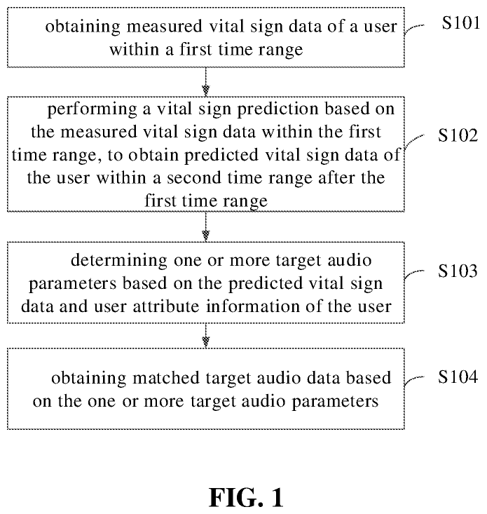

FIG. 1 is a flowchart illustrating an example of the method for audio playback provided in the present disclosure.

The method for audio playback is executed by the apparatus for audio playback, and the apparatus for audio playback can be applied to any type of electronic devices, such as a smart phone, a tablet computer, a vehicle mounted device, a smart audio, a wearable device, etc. For ease of understanding, the method is described below with an example of being executed by a wearable device.

As illustrated in FIG. 1, the method for audio playback includes the following operations S101 to S104.

At S101, measured vital sign data of a user within a first time range is obtained.

The first time range can be a certain moment, or a continuous period of time, such as 1 minute, 5 minutes, a duration of an audio, or a preset duration of time, or the first time range includes a plurality of moments distributed at a specific time interval. The first time range can be a preset time range starting from a moment when the user triggers audio playback, or a preset time range before the moment when the user triggers audio playback, or a preset time range after the moment when audio starts to be played. The duration of the first time range is set according to actual requirements.

The measured vital sign data includes at least one selected from heart rate data, activity amount data, physiological pressure data, sleep data, emotion data or respiratory rate data.

The measured vital sign data is collected by one or more sensors set in the wearable device, such as an acceleration sensor, a heart rate sensor, etc. A sensor set in the wearable device monitors the vital sign status of the user in real time, and the number of the sensors set in the wearable device is one or more.

In some implementations, the electronic device can obtain the measured vital sign data from at least one of a local sensor or local storage. In some implementations, the electronic device can obtain the measured vital sign data from other devices.

In some implementations, the measured vital sign data of the user can include a preset kind of data. In some implementations, the measured vital sign data of the user within the first time range is obtained based on user scenario information.

The user scenario information indicates the health requirements of the user.

Different user scenario information corresponds to different health requirements. The health requirements of the user include one or any combination of sleep aid, focus, weight loss or emotion regulation.

In some implementations, in response to the user scenario information indicating a sleep aid scenario or a focus scenario, heart rate data and activity data of the user within the first time range are obtained. In some implementations, in response to the user scenario information indicating a weight loss scenario, at least one of the activity data, motion data, the heart rate data, or positioning data of the user within the first time range is obtained. In some implementations, in response to the user scenario information indicating an emotion regulation scenario, heart rate variability of the user within the first time range is obtained.

In the case that the user scenario information indicates the sleep aid scenario or the focus scenario, the measured vital sign data includes the heart rate data and/or the activity data of the user. The heart rate data and the activity data can be used to represent a relaxation state of the user. In the case that the user scenario information indicates the weight loss scenario, the measured vital sign data can include, for example, the activity data, the motion data, the heart rate data, and the positioning data. The activity data, the motion data, the heart rate data, and the positioning data can be used to represent an activity status of the user, and can represent activeness, activity amount or the like of the user. In the case that the user scenario information indicates the emotion regulation scenario, the measured vital sign data can include, for example, the heart rate variability. The heart rate variability represents emotion state of the user.

In some implementations, vital sign data of the user at a target moment is collected.

In the implementation of the present disclosure, the vital sign data of the user can be collected by a sensor mounted in the wearable device. The sensor mounted in the wearable device monitors the current vital sign status of the user in real time.

In some implementations, the moment when the user triggers audio playback is taken as the target moment, and the vital sign data collected at the moment when the user triggers audio playback is taken as the vital sign data of the user at the target moment.

In some implementations, the next moment after the user triggers audio playback is taken as the target moment for data collection. The number of the sensors mounted in the wearable device is one or more.

As an example, the method for audio playback can be applied to a smart watch for illustrative explanation. The user is the person wearing the smart watch, and current vital sign state of the user is monitored in real time by the one or more sensors mounted in the smart watch. The target moment can include, for example, each moment after the user triggers the audio playback and the vital sign data of the user at the target moment is collected.

At S102, a vital sign prediction is performed based on the measured vital sign data within the first time range, and predicted vital sign data within a second time range after the first time range is obtained.

The second time range occurs after the first time range. The duration of the second time range can be the same as or different from that of the first time range.

In some implementations, the vital sign prediction is performed based on the measured vital sign data within the first time range, and the predicted vital sign data within the second time range is obtained. For example, the vital sign prediction can be performed based on a preset algorithm or formula. For another example, the vital sign prediction can be performed using a prediction model based on machine learning or deep learning. For instance, a feature extraction processing can be performed on the measured vital sign data within the first time range, to obtain first feature data, the first feature data and user attribute information are fused to obtain second feature data, and the vital sign prediction is performed based on the second feature data, to obtain the predicted vital sign data within a second time range.

The predicted vital sign data can include the same kind of vital signs as the measured vital sign data. Alternatively, the predicted vital sign data can include different kinds of vital signs as the measured vital sign data. The predicted vital sign data can include at least one of activity data, motion data, heart rate data, sleep data, emotion data, physiological stress data, respiratory rate data, or positioning data. For example, different kinds of predicted vital sign data are obtained in different scenarios. In some implementations, in response to the user scenario information indicating a sleep aid scenario or a focus scenario, heart rate data and activity data of the user within the second time range are obtained. In some implementations, in response to the user scenario information indicating a weight loss scenario, at least one of the activity data, motion data, the heart rate data, or positioning data of the user within the second time range is obtained. In some implementations, in response to the user scenario information indicating an emotion regulation scenario, heart rate variability of the user within the second time range is obtained. In some implementations, other kinds of the predicted vital sign data are obtained.

The user attribute information includes basic information of the user, which can include, for example, at least one of age, gender, height, weight or disease status. In some implementations, attributes of the user are related to the environment the user is in, and accordingly the user attribute information can also include environment information of the environment the user is in, where the environment information includes at least one of weather or time.

In some implementations, in the procedure, the vital sign prediction is performed based on the user vital sign data at the target moment, to obtain the predicted vital sign data at a set moment after the target moment.

In the implementations of the present disclosure, the vital sign prediction is performed on the user vital sign data at the target moment, and the predicted vital sign data at the set moment after the target moment is obtained by predicting the future natural evolution of the vital signs of the user at the target moment.

In some implementations, change patterns of the vital sign data, such as the heart rate, the activity amount and the pressure over time, are captured to perform the vital sign prediction.

At S103, one or more target audio parameters are determined based on the predicted vital sign data and the user attribute information of the user.

The one or more target audio parameters are determined based on the predicted vital sign data and the user attribute information of the user. In some examples, the predicted vital sign data and the user attribute information of the user can be processed by a model, such as a machine learning model or a deep learning model, so as to obtain the one or more target audio parameters. The input of the model can include at least one of: the predicted vital sign data and the user attribute information of the user, information obtained by processing at least one of the predicted vital sign data and the user attribute information, or other parameters, such as at least one physiological parameter of the user. The output of the model can include the one or more target audio parameters, or information for identifying the one or more target audio parameters, or a combination thereof.

In some other examples, the predicted vital sign data and the user attribute information of the user are mapped to the one or more target audio parameters. A mapping relationship between the vital sign data, the user attribute information, and the audio parameter can be predetermined, and the mapping relationship can be utilized to map the predicted vital sign data and the user attribute information of the user to the one or more target audio parameters. Alternatively, a plurality of mapping relationships between the vital sign data and the audio parameter can be set in advance. In this case, a target mapping relationship is determined from the plurality of mapping relationships based on the user attribute information of the user, and the target mapping relationship is utilized to map the predicted somatic data to the one or more target audio parameters. In one example, a plurality of user groups are determined, and each user group are associated with one mapping relationship between the vital sign data and the audio parameter. A target user group to which the user belongs can be determined based on the user attribute information of the user, and the predicted vital sign data is mapped to the one or more target audio parameters by utilizing the mapping relationship associated with the target user group.

In some other examples, at least one predicted physiological parameter of the user is determined based on the predicted vital sign data of the user, and the predicted physiological parameter and the user attribute information of the user are used to determine the one or more target audio parameters.

In some implementations, at least one target physiological parameter of the user is determined, and the one or more target audio parameters are determined based on the predicted vital sign data, the at least one target physiological parameter and the user attribute information. The at least one target physiological parameter is used to indicate the type of physiological parameter, alternatively, the target physiological parameter is used to indicate not only the type of physiological parameter, but also the target value of the physiological parameter. In one example, the at least one target physiological parameter of the user includes at least one of a target activeness, a target activity amount, a target heart rate, a target heart rate variability, or the like.

In some implementations, the at least one target physiological parameter is determined based on at least one of the user scenario information, current physiological parameter of the user, historical vital sign data of the user or the user attribute information of the user. Taking determining the at least one target physiological parameter of the user based on the user scenario information for example, in response to the user scenario information indicating the sleep aid scenario or the focus scenario, a target activeness of the user is determined. For example, if the scenario set by the user is the weight loss scenario, the corresponding target physiological parameter includes a target activeness, which is used as a measure of relaxation of the user before going to sleep. In this case, taking the activeness of the user as the target physiological parameter is helpful for the user to enter a sleep state as soon as possible.

In another instance, in response to the user scenario information indicating the weight loss scenario, at least one of a target activeness, target activity amount or a target heart rate of the user is determined. For example, in the weight loss scenario, the target activeness, the target activity amount and the target heart rate of the user are determined. The target heart rate is the heart rate for the user achieving an effect of burning fat, and it can be determined based on the user attribute information of the user, such as the weight, height or the like. The target activity amount can be set by the user or determined based on at least one of historical activity data of the user, the current weight or the target weight of the user.

In another instance, in response to the user scenario information indicating an emotion regulation scenario, a target heart rate variability of the user is determined. If the scenario set by the user is the emotion regulation scenario, the target physiological parameter includes HRV (heart rate variability). The emotion is highly correlated with HRV. A low HRV indicates a relaxed state, while a high HRV indicates a potential state of nervousness or depression. Taking HRV as the target physiological parameter can help the user adjust emotion in a timely manner. In some implementations, when it is detected that the user is in a nervous or depressed state, a lower HRV is set as the target physiological parameter. For example, a HRV lower than a preset threshold is determined, or a HRV lower than the current HRV of the user is determined. The target heart rate variability of the user can be a preset value, or has a fixed step from the current HRV of the user, or has a step from the current HRV depending on the duration of the second time range.

In some implementations, the target physiological parameter of the user is determined based on at least one of the user scenario information and historical vital sign data of the user. The historical vital sign data can include vital sign data of the user before the first time range. For example, the historical vital sign data of the user includes vital sign data of the user within a third time range before the first time range. The third time range can be adjacent to the first time range, or has an interval from the first time range. Alternatively, the historical vital sign data can include vital sign data of the user before the second time range. For example, the historical vital sign data of the user includes the vital sign data of the user within the first time range.

There are various manners to obtain the historical vital sign data of the user. For example, the historical vital sign data of the user can be obtained from a local storage space, or from another device.

In some implementations, the user scenario information is used for determining the category of the physiological parameter. For example, in a sleep aid scenario, the physiological parameter is determined to include activeness of the user. For another example, in a weight loss scenario, the physiological parameter is determined to include activity amount and heart rate of the user. The historical vital sign data of the user is used for determining the historical value of the physiological parameter. Then, the target physiological parameter, i.e., the target value of the physiological parameter, is determined based at least part on the historical value of the physiological parameter.

In some implementations, the target physiological parameter includes the activeness. The target physiological parameter of the user is determined based on at least one of the user scenario information and the historical vital sign data of the user. For example, historical heart rate data and historical activity data of the user within a third time range corresponding to the first time range is obtained, and historical activeness of the user is determined based on the historical heart rate data and the historical activity data of the user. The target activeness of the user is determined based on the historical activeness of the user, where the target activeness is less than the historical activeness. The target activeness can be less than the historical activeness by a step, or the target activeness is chosen from a plurality of preset values of activeness based on the historical activeness, or the target activeness is obtained from a preset algorithm or a preset model based on the historical activeness. In this case, the historical vital sign data includes the historical heart rate data and the historical activity data. The historical vital sign data is the data of the user stored in the wearable device, or in an electronic device providing the audio to the user, or in a server.

There are various implementations to determine the one or more target audio parameters based on the predicted vital sign data, the at least one target physiological parameter and the user attribute information. In some examples, at least one predicted physiological parameter is determined based on the predicted vital sign data, a difference between the at least one predicted physiological parameter and the at least one target physiological parameter is determined, and one or more target audio parameters are determined based on the difference and the user attribute information of the user. In some examples, the predicted vital sign data, the target physiological parameter and the user attribute information are inputted into a neutral network model or a regression model, and the one or more target audio parameters are outputted by the neutral network model or the regression model.

The one or more target audio parameters include at least one of audio loudness, rhythm, tempo, key, chords, drum beats, melodies, natural notes, soundscapes or the like. In one example, the one or more target audio parameters include target audio loudness and target rhythm.

In the implementations of the present disclosure, due to the different requirements for adjusting vital signs in different scenarios, the corresponding target physiological parameters are different in different scenarios. In some implementations, if the scenario set by the user is a weight-loss-by-exercise scenario, the corresponding target physiological parameter includes a fat burning heart rate for the user in an exercise state. The fat burning heart rate is one of metrics for measuring weight loss by exercise, and taking the fat burning heart rate as the target physiological parameter can be helpful for the user to obtain higher weight loss efficiency.

In some implementations, in the case that the scenario is set as the weight-loss-by-exercise scenario, for users not in the exercise state, for example, the heart rate of the user is lower than or within a preset heart rate range, rhythmic audio can be provided to the user, so as to improve the user's interest in exercise. In some implementations, in combination with a sedentary reminder function of the wearable device, in response to detecting that the user is sedentary, rhythmic audio is provided to guide the user to do some exercises. In some implementations, audio suitable for outdoor activities is provided to guide the user to perform outdoor activities.

In some implementations, there is one or more mapping relationships between the vital sign data, at least one physiological parameter and user attribute information with at least one audio parameter. In this case, the one or more target audio parameters are determined by querying the mapping relationship based on the predicted vital sign data, the target physiological parameter and the user attribute information of the user, and thus, the one or more target audio parameters are matched with the predicted vital sign data, the target physiological parameter and the user attribute information of the user.

At S104, matched target audio data is obtained based on the one or more target audio parameters.

In some implementations, an audio library with a plurality of audios can be established in advance, and each audio is associated with one or more audio parameters. Alternatively, an audio parameter library with a plurality of audio parameter combinations can be established in advance. The one or more target audio parameters can be used to look up in the audio parameter library, so as to obtain the target audio parameter combination that matches the target audio parameters. For example, it is determined whether at least one audio parameter associated with an audio matches with the one or more target audio parameters, so as to determine if the audio is the target audio. For another example, it is determined whether at least one audio parameter associated with an audio parameter combination matches with the target audio parameters, so as to determine a target audio parameter combination for generating the audio to be provided to the user in real time.

In some implementations, the one or more target audio parameters include one or more target audio modulation parameters, and the matched target audio data is obtained based on the one or more target audio modulation parameters. The audio modulation parameters can be obtained by modulating at least one original audio element, e.g., drum beats, melodies, natural notes, soundscapes, etc. The modulation can be performed by using different audio parameters, such as, for example, loudness and/or rhythm. In this case, the audio modulation parameters can include modulated drum beats, modulated melodies, modulated natural notes, modulated soundscapes and/or the like. Alternatively, the audio modulation parameters can include audio parameters for modulating the original audio elements. For example, the audio modulation parameters include loudness, rhythm and/or the like.

In one example, the one or more audio modulation parameters are used to generate the audio. In another example, the one or more target audio modulation parameters are sent from the wearable device to a server, the server queries the target audio data matching the one or more target audio modulation parameters, and the target audio data sent by the server is obtained by the wearable device.

The target audio modulation parameters may include the target audio loudness and the target rhythm.

In some implementations, a target audio can be obtained and provided to the user according to the one or more target audio modulation parameters. For example, the target audio has audio characteristics matching with the one or more target audio parameters. For another example, the target audio is provided to the user within the second time range.

In the implementation of the present disclosure, audio elements are modulated and classified in advance, and an audio modulation library is generated and stored at the cloud side. The audio elements include a plurality of combinations of drum points, melodies, natural sounds and loudness.

In some implementations, matched audio elements are queried based on the target audio modulation parameters, namely the target audio loudness and the target rhythm, queried audio elements are synthesized to obtain the target audio, and the target audio is played.

Based on the above, the measured vital sign data of the user within the first time range is obtained. The vital sign prediction is performed based on the measured vital sign data within the first time range, to obtain the predicted vital sign data of the user within the second time range after the first time range. The one or more target audio parameters are determined based on the predicted vital sign data, the target physiological parameter and the user attribute information, and the matched target audio data is obtained based on the one or more target audio parameters. Since the one or more target audio parameters are determined based on the predicted vital sign data, the target physiological parameter and the user attribute information, the target audio data matched with the one or more target audio parameters can be effective for adjusting sleep or health for the user, thereby solving the technical problem of the related technologies that actual audio playback does not match the requirements of the user and the adjustment effect is poor.

In another aspect of the present disclosure, another method for audio playback is provided. Specifically, the target physiological parameter of the user is determined based on the user scenario information, where the user scenario information is used for indicating health requirements of the user, one or more target audio parameters are determined based on the target physiological parameter of the user, and matched target audio data is obtained based on the one or more target audio parameters.

According to the method for audio playback in the implementations of the present disclosure, the target physiological parameter is determined based on user scenario information, one or more target audio parameters are determined based on the target physiological parameter of the user, and matched target audio data is determined based on the one or more target audio parameters. The user scenario information is used for indicating the health requirements of the user. In this way, starting from the health needs of the user, the one or more target audio parameters that enable the user to achieve the target physiological parameter are quantified by a model for matching the target physiological parameter and the one or more target audio parameters. The manner of active modulation effectively determines the target audio based on the health requirements of the user, thereby improving health status of the user more effectively.

One or more target physiological parameters are determined based on the user scenario information. In some implementations, the user scenario information is used for determining the parameter category of the target physiological parameter, i.e., the user scenario information is used for determining which kind of parameter is included in the target physiological parameter. For example, in response to the user scenario information indicating the sleep aid scenario or the focus scenario, the target physiological parameter includes an activeness of the user. For another example, in response to the user scenario information indicating the weight loss scenario, the target physiological parameter includes at least one of an activeness, an activity amount or a heart rate of the user. For another example, in response to the user scenario information indicating the emotion regulation scenario, the target physiological parameter includes a heart rate variability of the user. In some implementations, the user scenario information is used for determining not only the parameter category of the target physiological parameter, but also a value of the target physiological parameter. In one example, the target physiological parameter of the user is determined based on the user scenario information and at least one of current physiological parameter of the user, historical vital sign data of the user or the user attribute information of the user. The above description can be referred to for more details.

In some implementations, one or more target audio parameters are determined based on the target physiological parameter of the user and at least one of user attribute information and vital sign data of the user. The vital sign data of the user may include vital sign data that is measured by at least one sensor, or the vital sign data of the user may include vital sign data that is predicted according to the measured vital sign data of the user.

The category of the vital sign data can be preset. In some implementations, the vital sign data is determined based on the user scenario information. For example, in the case that the user scenario information indicates the sleep aid scenario or the focus scenario, the vital sign data can include the heart rate data and/or the activity data of the user. The heart rate data and the activity data can be used to represent a relaxation state of the user. For another example, in the case that the user scenario information indicates the weight loss scenario, the vital sign data can include at least one of activity data, motion data, the heart rate data, or positioning data. The activity data, the motion data, the heart rate data, and the positioning data can be used to represent an activity status of the user, which can be used to represent an activeness, an activity amount of the user or the like. For another example, in the case that the user scenario information indicates the emotion regulation scenario, the vital sign data can include, for example, the heart rate variability. The heart rate variability can be used to represent an emotional state of the user.

In some implementations, one or more target audio parameters are determined based on the target physiological parameter of the user and the vital sign data of the user, where the vital sign data of the user includes measured vital sign data of the user within a first time range. Specifically, measured vital sign data of the user within the first time range is determined based on the user scenario information, a vital sign prediction is performed based on the measured vital sign data of the user within the first time range, so as to obtain predicted vital sign data of the user within the second time range, and the one or more target audio parameters are determined based on the predicted vital sign data and the target physiological parameter.

The duration of the second time range can be the same as or different from that of the first time range.

In some implementations, the measured vital sign data of the user within the first time range is determined based on the user scenario information. Users usually have different requirements for listening to the audio in different scenarios, and thus different kinds of vital sign data are obtained in different scenarios, which in turn is used for choosing the proper audio for the user, so as to better achieve the requirements of the user for audio playback in different scenarios. In some examples, in response to the user scenario information indicating the sleep aid scenario or the focus scenario, the heart rate data and the activity data of the user within the first time range is obtained. In some examples, in response to the user scenario information indicating the weight loss scenario, at least one of the activity data, the motion data, the heart rate data, or the positioning data of the user within the first time range is obtained. In some examples, in response to the user scenario information indicating an emotion regulation scenario, the heart rate variability of the user within the first time range is obtained.

The vital sign prediction is performed based on the measured vital sign data within the first time range, so as to obtain the predicted vital sign data within the second time range after the first time range. The vital sign prediction can be performed by a vital sign predication model, and the vital sign prediction model can be based on an algorithm, a machine learning model or a deep learning model, such as a neural network model. In an example, feature extraction processing is performed on the measured vital sign data within the first time range, to obtain first feature data. The first feature data and the user attribute information are fused to obtain second feature data. The prediction is performed based on the second feature data, to obtain the predicted vital sign data within the second time range.

The user attribute information includes the basic user information, which can include at least one of the age, the gender, the height, the weight or the health status. In some implementations, the user attributes are related to the environment the user is in, and thus the user attribute information also includes the environment information of the environment the user is in, and the environment information includes at least one of weather or time.

In some implementations, the one or more target audio parameters are determined based on the predicted vital sign data, the target physiological parameter and user attribute information. For example, the predicted vital sign data, the target physiological parameter and the user attribute information are inputted into the regression model, to obtain the one or more target audio parameters outputted by the regression model.

The one or more target audio parameters can include the target audio loudness and the target rhythm.

In some implementations, the one or more target audio parameters can include the target audio modulation parameters.

The matched target audio data can be obtained based on the one or more target audio parameters. Specifically, the target audio modulation parameters can be sent to the server, so as to enable the server to query target audio data matching the target audio modulation parameter; and the target audio data sent by the server is obtained.

An example is described below to illustrate the above implementations.

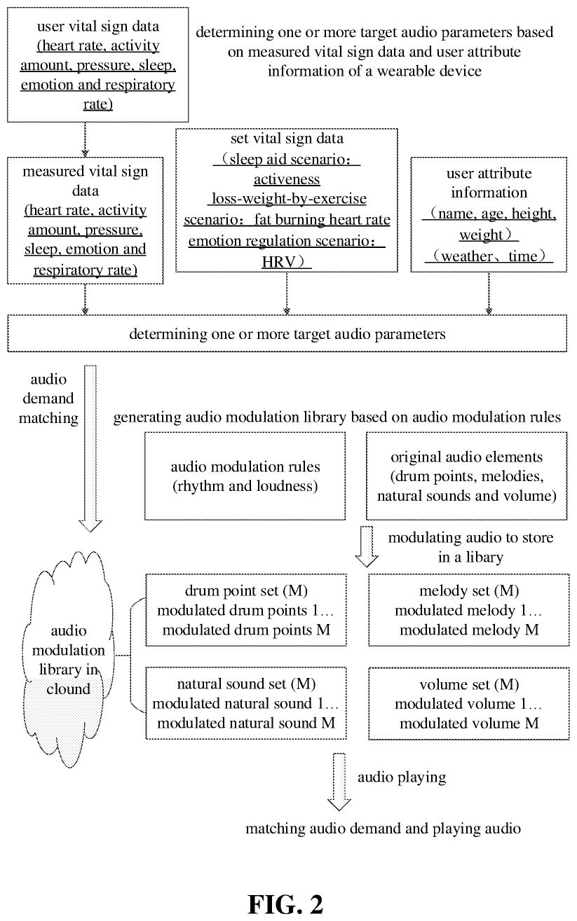

In the example as illustrated in FIG. 2, vital sign data of a user is measured by at least one sensor. The vital sign data of a user includes at least one of heart rate data, activity amount data, pressure data, sleep data, emotion data or respiratory rate data. The vital sign data of the user is measured and used to obtain predicted vital sign data.

The predicted vital sign data includes one or more of the heart rate data, the activity amount data, the pressure data, the sleep data, the emotion data or the respiratory rate data. The predicted vital sign data can have the same or different kinds of data as the vital sign data of the user.

User scenario information for indicating current scenario is obtained. In some examples, the current scenario is set by the user, and the user scenario information can be obtained from the user input. In some examples, the user scenario information can be obtained based at least in part on sensed data from at least one sensor, such as, for example, positioning sensor, motion sensor, environment light sensor, sound sensor or the like. Then, one or more target physiological parameters are determined according to the user scenario information. For different scenarios, target physiological parameters can be different. For example, if a scenario set by the user is a sleep aid scenario, the corresponding target physiological parameter includes activeness. For example, if a scenario set by the user is a weight-loss-by-exercise scenario, the corresponding target physiological parameter can include, for example, fat burning heart rate. For example, if a scenario set by the user is an emotion regulation scenario, the corresponding target physiological parameter can include, for example, HRV.

User attribute information of the user is also obtained. The user attribute information includes basic information of the user, which includes at least one of age, gender, height, weight or disease status. In some implementations, attributes of the user are related to the environment the user is in, and accordingly the user attribute information also includes the environment information of the environment the user is in, where the environment information includes at least one of weather or time.

The predicted vital sign data, the target physiological parameters and the user attribute information are used to determine at least one target audio modulation parameter. The audio modulation parameter can include audio loudness and rhythm.

The at least one target audio modulation parameter is used to obtain target audio data. There can be a plurality of audio modulation sets in an audio modulation library, each corresponds to a combination of original audio elements and at least one audio modulation parameter, where the original audio elements include drum points, melodies, natural sounds and loudness. The original audio elements can be modulated with an audio modulation rule by using at least one audio modulation parameter to obtain the plurality of audio modulation sets.

For example, as illustrated in FIG. 2, the method for audio playback can be implemented in three parts. In the first part, one or more target audio modulation parameters can be determined based on measured vital sign data and the user attribute information of the user. Specifically, natural evolution of vital sign of the user in the upcoming future is predicted based on vital sign data of the user measured in real time, so as to obtain the predicted vital sign data. One or more target physiological parameters corresponding to the user scenario information are obtained, where the user scenario information indicates the current scenario such as a sleep aid scenario, a weight-loss-by-exercise scenario, or an emotion regulation scenario. The target physiological parameter corresponding to the sleep aid scenario optionally includes activeness, the target physiological parameter corresponding to the weight-loss-by-exercise scenario optionally includes fat burning heart rate, and the target physiological parameter corresponding to the emotion regulation scenario optionally includes HRV The user attribute information obtained via the wearable device includes basic information of the user. The basic information of the user includes at least one of the age, the gender, the height, the weight or the disease status. Based on the environment the user is in, the wearable device further obtains environment information of the environment the user is in. The environmental information includes at least one of weather or time. Finally, the one or more target audio modulation parameters are determined based on the predicted vital sign data, the target physiological parameter and the user attribute information. For example, the predicted vital sign data, the target physiological parameter and the user attribute information are input into a deep learning model, such as a multilayer residual network model, or into a machine learning model, such as XGBoost (eXtreme Gradient Boosting) or an SVR (Support Vector Regression) model, so as to obtain the one or more target audio modulation parameters based on the output of the model.

In the second part, an audio modulation library can be generated based on an audio modulation rule, that is, the original audio elements are modulated based on the audio modulation rule, to generate the audio modulation library, and the audio modulation library can be stored at the cloud server. The parameter for the audio modulation rule includes rhythm and loudness, and the original audio elements include drum points, melodies, natural sounds and loudness with various rhythms and loudness. In some implementations, the original audio elements are modulated with different versions of rhythms and loudness with high, medium and low proportion, to obtain a plurality of audio modulation sets, such as a modulated drum point set, modulated melody set, modulated natural note set and modulated soundscape set. It should be understood that the audio modulation library can be generated in advance, that is, the audio modulation library can be generated before the matching of required audio is performed.

In the third part, the obtained one or more target audio modulation parameters are used to determine a matched audio modulation set, which in turn is used to generate the required audio in real time and then provided to the user. That is, appropriate audio modulation set is matched from the audio modulation library at the cloud server based on the one or more target audio modulation parameters determined in the first part, and the audio is synthesized and played for the user by using the audio modulation set, so as to finally achieve a positive guidance for the user's sleep or health.

Based on the above, the natural evolution of vital signs of the user in the upcoming future is predicted based on the vital sign data measured in real time by the wearable device, to obtain predicted vital sign data. The target physiological parameter corresponding to user scenario information can be obtained based on the user scenario information of audio set by the user. The user attribute information can be obtained by the wearable device, so that the one or more target audio parameters are determined based on the predicted vital sign data, the target physiological parameter and the user attribute information. Then the appropriate audio modulation set is matched from the audio modulation library in the cloud server based on the one or more target audio modulation parameters, and the appropriate audio is generated and played. Since the one or more target audio parameters are determined based on the predicted vital sign data, the target physiological parameter and the user attribute information, the target audio matched with the one or more target audio parameters can be effective for adjusting sleep or health for the user, so as to solve the technical problem of the related technologies that actual audio playback does not match the user's requirements and the adjustment effect is poor.

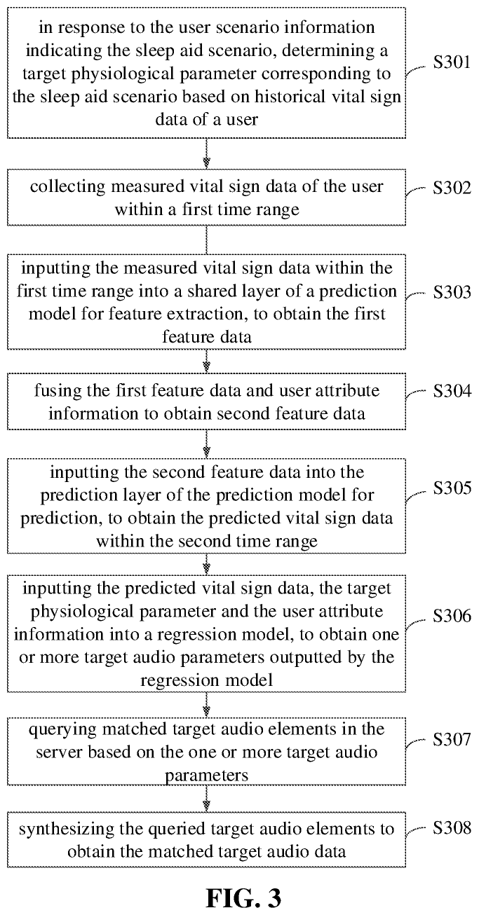

Another method for audio playback is provided to further illustrate implementations of the disclosure. FIG. 3 is a flowchart of another method for audio playback provided in another implementation of the disclosure.

As illustrated in FIG. 3, the method for audio playback includes the following operations S301 to S308.

At S301, in response to user scenario information indicating the sleep aid scenario, the target physiological parameter corresponding to the sleep aid scenario is determined based on historical vital sign data of a user.

Specifically, in the case that it is determined to be the sleep aid scenario based on the operation of the user, the target physiological parameter corresponding to the sleep aid scenario is determined based on the historical vital sign data of the user.

In some implementations, vital sign data of the user collected in real time is stored in a database or other storage space. The vital sign data stored is taken as the historical vital sign data, and the target physiological parameter corresponding to the sleep aid scenario is determined periodically based on the historical vital sign data. Since the target physiological parameter is determined periodically based on continuous real-time vital sign data of the user, its accuracy is relatively high, and target audio data obtained can effectively regulate user's sleep. Therefore, in the case of a relatively high demand for an accuracy of the target audio, periodic execution of the method can be adopted.

In some implementations, in response to entering the sleep aid scenario, the target physiological parameter corresponding to the sleep aid scenario is determined based on the historical vital sign data. Since the target physiological parameter is determined immediately when entering the sleep aid scenario, the execution efficiency is high, and the target audio data is quickly obtained and sleep adjustment is performed. Therefore, in the case of a high demand for the execution efficiency of the target audio, immediate execution responsive to entering into the sleep scenario can be adopted.

In the following examples of the present disclosure, the process of determining the target physiological parameter corresponding to the sleep aid scenario based on the historical vital sign data is described more specifically. The historical vital sign data of the user can be obtained by the sensor mounted in the wearable device, where the historical vital sign data includes historical heart rate data and historical activity amount data. Then, the historical heart rate data and the historical activity amount data can be normalized based on an average heart rate before sleep and an average activity amount obtained from the historical vital sign data of the user, so as to obtain normalized historical heart rate data HRₙₒᵣₘ and normalized historical activity amount data Activityₙₒᵣₘ, which can reflect the characteristics of heart rate and activity for each user individual. Then, in order to comprehensively reflect the impact of heart rate and activity amount, the normalized historical heart rate data and the normalized historical activity amount data can be weighted based on the following formula to determine historical activeness Activeness. The formula is as follows:

$$Activeness = {α}^{*} H {R}_{norm} + ( 1 - α ) * {Activity}_{n o r m} \qquad ( 1 )$$

where α is the weight of the historical heart rate data HRₙₒᵣₘ.

Finally, one or more target physiological parameters corresponding to the sleep aid scenario can be determined according to an activeness interval to which the historical activeness belongs. The activeness interval is optionally divided based on the activeness before sleep of the user wearing the wearable device. In some implementations, the quartiles Q1, Q2 and Q3 are calculated by analyzing distribution of the historical activeness before sleep of the user wearing the wearable device. The quartile, also known as quartet, refers to values at positions of three segmentation points in statistics when all values sorted in an ascending order are divided into four equal parts. The activeness before sleep is discretized into three quantitative levels based on the quartiles. In one illustrative example, the activeness before sleep less than Q1 is taken as a low activeness interval, the activeness before sleep within the range of [Q1, Q3] is taken as a medium activeness interval, and the activeness before sleep greater than Q3 is taken as a high activeness interval.

In some implementations, if the user is in the high activeness interval, the target physiological parameter can be set to be in the medium activeness interval. When the user is in the middle activeness interval, the target physiological parameter can be set to be in the low activeness interval.

At S302, measured vital sign data of the user within a first time range is collected.

In the implementation of the present disclosure, the measured vital sign data can include, for example, monitored data of heart rate and monitored data of activity amount.

The execution of S101 in the above implementations can be referred to for more details, which will not be repeated herein.

At S303, the measured vital sign data within the first time range is processed with a shared layer of a vital sign prediction model for a feature extraction, to obtain first feature data.

The measured vital sign data includes the measured heart rate data and the measured activity amount data.

The measured heart rate data within the first time range, per se or after being pre-processed, is inputted into a shared layer of the vital sign prediction model for feature extraction, to obtain the first feature data corresponding to the heart rate. The measured activity amount data within the first time range, per se of after being pre-processed, is inputted into a shared layer of the vital sign prediction model for feature extraction, to obtain the first feature data corresponding to the activity amount.

It should be noted that, the measured heart rate data and the measured activity amount data can be inputted into the same shared layer or different shared layers of the vital sign prediction model.

At S304, the first feature data and user attribute information are fused to obtain second feature data.

In this example, the user attribute information includes basic information of the user. The basic information of the user includes at least one of age, gender, height, weight or disease status. In some implementations, attributes of the user are related to the environment the user is in, and accordingly, the user attribute information also includes environment information of the environment the user is in, where the environment information includes at least one of weather or time.

In some implementations, the first feature corresponding to the heart rate and the first feature data corresponding to the activity amount can be fused with the user attribute information together, to obtain the second feature data. In some implementations, the first feature corresponding to the heart rate and the first feature data corresponding to the activity amount can be fused with the user attribute information separately, to obtain the second feature corresponding to the heart rate and the second feature data corresponding to the activity amount.

In some implementations, the first feature data corresponding to the heart rate and the first feature data corresponding to the activity amount obtained in S303 are spliced with the user attribute information to obtain the second feature data. Since the second feature data is obtained by directly splicing the first feature data and user attribute information, the execution efficiency is high, and the second feature data can be obtained quickly. Therefore, in the case that the predicted vital sign data needs to be obtained quickly, the manner of direct splicing can be adopted.

In some implementations, the first feature data corresponding to the heart rate and the first feature data corresponding to the activity amount obtained in S303 are fused by weight with the user attribute information to obtain the second feature data. Weights for the first feature data corresponding to the heart rate, the first feature data corresponding to the activity amount and user attribute information can be pre-configured, and there is a certain correlation between their weights and the degree of their influence on the predicted vital sign data. Specific values of the weights may be obtained by a limited number of tests. Among different values of the weights, the values which result in the most accurate value of the predicted vital sign data can be used for the fusion of the first feature data corresponding to heart rate, the first feature data corresponding to activity and user attribute information in a weighted manner. Since the second feature data is obtained by the fusion of the first feature data and user attribute information in a weighted manner, and the weights need to be obtained by a plurality of experiments, it has relatively high accuracy and can bring more accurate value for the predicted vital sign data. Therefore, the fusion in a weighted manner as described above can be used in the case that accurate prediction of the vital sign data is required.

At S305, the second feature data is inputted into the prediction layer of the vital sign prediction model for vital sign prediction, to obtain the predicted vital sign data within the second time range.

In some implementations, the second feature data corresponding to the heart rate is inputted into a prediction layer of the vital sign prediction model for heart rate prediction, so as to obtain the prediction heart rate data, and the second feature data corresponding to the activity amount is inputted into a prediction layer of the vital sign prediction model for activity amount prediction, so as to obtain the prediction activity amount data. The prediction layer for the heart rate prediction can be the same or different from the prediction layer for the activity amount prediction. In some implementations, the second feature data as a whole is inputted into a prediction layer of the vital sign prediction model for vital sign prediction, so as to obtain the predicted heart rate data and the predicted activity amount data.

At S306, the predicted vital sign data, the target physiological parameter and the user attribute information are processed with a regression model, to obtain one or more target audio parameters.

In some implementations, the regression model can be a trained model, which has learned a mapping relationship between the predicted vital sign data, the target physiological parameter and user attribute information with the one or more target audio parameters.

The user attribute information includes the basic information of the user, which includes at least one of the age, the gender, the height, the weight or the disease status.

In some implementations, user's attributes are related to the environment the user is in, so the user attribute information can further include the environment information of the environment the user is in, and the environment information includes at least one of weather or time. For example, the weather is sunny, cloudy, etc., and the time is related to the target moment, such as, for example, at least a part of the day, month, hour, minute, or second. The time can include, for example, only hour, minute, and second. The choice of time granularity is determined according to the prediction accuracy. Generally, there is a periodic change in emotion of the user within a day. For example, in the morning, the psychological pressure of the user is usually low and gradually becomes intense over time, and the psychological pressure has a decline trend near noon. Similarly, due to different activities carried out by the user, attributes of the user in different months or seasons within a year show different characteristics. Therefore, in order to accurately predict the user's demand, the appropriate time granularity can be selected according to the prediction accuracy. In the case of a relatively high demand for prediction accuracy, combinations of more time granularities can be selected, such as day and month, as well as hour, minute and second. In the case of a relatively low demand for prediction accuracy, a combination with less time granularities can be selected, such as hour and minute.

In this example, the one or more target audio parameters include target audio loudness and target rhythm. Loudness of audio refers to the intensity of the sound sensed by the ear, and it is a subjective perception of the volume of the sound. Rhythm refers to a regular mutation that accompanies rhythm in natural, social and human activities.

In some implementations, before the predicted vital sign data, the target physiological parameter and the user attribute information are inputted into the regression model, a feature engineering processing needs to be performed on the predicted vital sign data, the target physiological parameter and the user attribute information, so as to convert the data into input that is suitable for the regression model to process.

The regression model can be obtained by the process of model training. In some implementations, a training process can be performed before the wearable device is delivered from the factory. For example, the regression model can be trained by vital sign data of test users collected offline, and the regression model is loaded into the wearable device after it is trained. In some implementations, during usage of users, the vital sign data of the users is recorded, and one or more historical data samples are generated based on the audio selected by the user's audio selection operations, and the regression model can be trained by using this data to obtain a regression model with a better adjustment effect.

At S307, matched target audio elements are queried in a server based on the one or more target audio parameters.

In this implementation, original audio elements are modulated and classified to obtain a plurality of modulated audio elements, and an audio modulation library is generated and stored in the server. The original audio elements can include a plurality of combinations of drum points, melodies, natural notes and soundscapes.

In some implementations, the matched target audio elements are queried in the server based on the one or more target audio parameters, i.e., the target audio loudness and the target rhythm. Modulated audio elements associated with a plurality of values of the rhythm and loudness are stored in the server. These modulated audio elements can be obtained in advance, for example, by processing the audio in an audio set. In some implementations, the audio in the audio set can be modulated according to an audio modulation rule. For example, the drum points and the melodies are main parts of the audio for adjusting the emotion of the user and rhythm. The natural note and soundscape are used as auxiliary parts for creating the environment atmosphere. Therefore, the drum points, the melodies, the natural notes and soundscape are modulated into different rhythm and loudness versions with high, medium and low modulation proportion according to the audio modulation rule, to obtain modulated drum point set, melody set, natural note set and soundscape set, and the audio modulation library is generated and stored in the server.

At S308, the queried target audio elements are synthesized to obtain the target audio data.

In this operation, by obtaining the target audio data, the target audio for the user can be played on the wearable device or other electronic devices.

In some implementations, the matched target audio elements queried in the server are synthesized to obtain the target audio data.

In some implementations, the modulated drum points, the melodies, the natural notes and the soundscape associated with the target audio loudness and the target rhythm that are obtained in the previous operation, such as S307, can be inputted into different audio tracks for synthetization, so as to obtain the target audio for play.

Based on the above, the measured vital sign data within the first time range is collected. The vital sign prediction is performed based on the measured vital sign data within the first time range to obtain the predicted vital sign data of the user within the second time range after the first time range. The one or more target audio parameters are determined based on the predicted vital sign data, the target physiological parameter and the user attribute information. And the matched target audio data is obtained based on the one or more target audio parameters. Since the one or more target audio parameters are determined based on the predicted vital sign data, the target physiological parameter and the user attribute information, the target audio data matched with the one or more target audio parameters can be effective for adjusting sleep or health for the user, so as to solve the technical problem of the related technologies that actual audio playback does not match requirements of the user and the adjustment effect is poor.

The following example illustrates the acquisition of the predicted vital sign data.

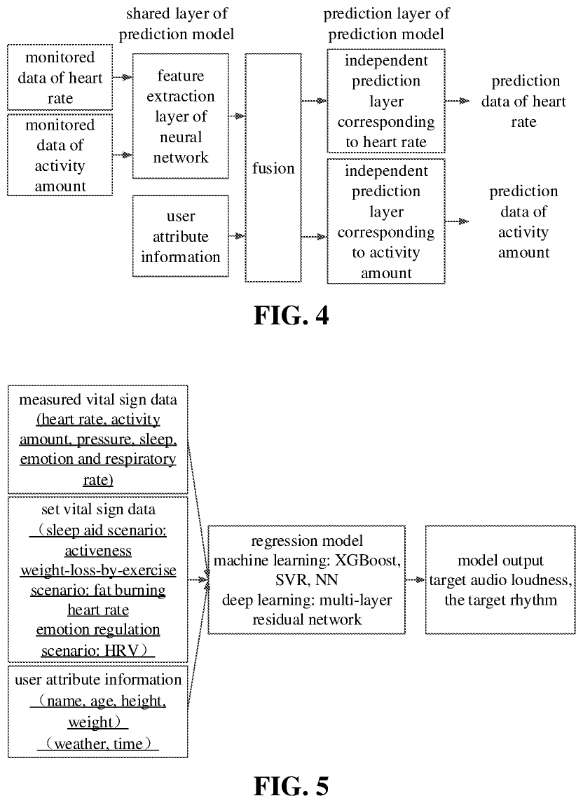

For example, the vital sign prediction model shown in FIG. 4 captures change patterns of vital sign data such as a heart rate, activity amount and a physiological pressure over time by using multi-task learning technologies in deep learning, so as to perform the vital sign prediction. Taking the heart rate and the activity amount as an example, the monitored data of heart rate and the monitored data of activity amount are inputted into a feature extraction layer of the shared layer in the neural network prediction model, to extract feature data, so that the first feature data corresponding to heart rate and the first feature data corresponding to activity amount are obtained. The monitored data of heart rate and the monitored data of activity amount are obtained via the wearable device. Then the first feature data and user attribute information are fused, that is, the first feature data corresponding to heart rate and the first feature data corresponding to activity amount are spliced with the user attribute information respectively, to obtain second feature data corresponding to heart rate and the second feature data corresponding to activity amount. Finally, the second feature data is inputted into the prediction layer of the neural network prediction model for vital sign prediction, that is, the second feature data corresponding to heart rate and the second feature data corresponding to activity amount are respectively inputted into a prediction layer corresponding to the heart rate and another prediction layer corresponding to activity amount for vital sign prediction, so as to obtain the prediction data of heart rate and the prediction data of activity amount.

In this example, the monitored data of heart rate and the monitored data of activity amount are inputted into a same shared layer of the prediction model.

In this example, the first feature data corresponding to heart rate and the first feature data corresponding to activity amount are spliced with the user attribute information respectively, so as to obtain the second feature data. In some other implementations, the first feature data corresponding to heart rate and the first feature data corresponding to activity amount are fused with the user attribute information in a weighted manner, so as to obtain the second feature data.

Based on the above, the changing patterns of heart rate and activity amount over time can be adequately captured to obtain corresponding predicted vital sign data.

The following example illustrates determination of the one or more target audio parameters.

For example, as illustrated in FIG. 5, a regression model is used. Predicted vital sign data, target physiological parameter and user attribute information are inputted into the regression model, to obtain one or more target audio parameters outputted by the regression model. The predicted vital sign data includes one or more combinations of heart rate data, activity amount data, physiological pressure data, sleep data, emotion data or respiratory rate data.

The user attribute information includes basic information of the user, which includes at least one of age, gender, height, weight or disease status. In some implementations, the user attribute information further includes environment information of the environment the user is in, and the environment information includes at least one of weather or time.

The one or more target audio parameters include target audio loudness and target rhythm.

In some implementations, the regression model is a trained model and has learned a mapping relationship between the predicted vital sign data, the target physiological parameter and the user attribute information with the one or more target audio parameters. The regression model can be selected from a machine learning regression model such as, for example, a XGBoost (extreme gradient boosting) model, a SVR (support vector regression) model, a NN (neural network) model, or a deep learning regression model such as a multi-layer residual network model.

In this example, feature engineering can be firstly performed on the predicted vital sign data, the target physiological parameter and the user attribute information, so as to convert them into data that is suitable for the regression model to process.

In some implementations, the gender information in the basic user information and the weather information in the environment information are processed as categorical data with single-hot coding, and other information as numerical data with min-max normalization or Gaussian normalization.

Further, data processed by feature engineering, i.e., the predicted vital sign data, the target physiological parameter and the user attribute information are inputted into the regression model, to obtain the one or more target audio parameters, i.e., the target audio loudness and the target rhythm.

Based on the above, by inputting the data processed by feature engineering, e.g., the predicted vital sign data, the target physiological parameter and the user attribute information, into the regression model, the one or more target audio parameters outputted by the regression model are obtained. As a result, the target audio data matched with the one or more target audio parameters can effectively adjust sleep or health for the user, thereby solving the technical problem of the related technologies that actual audio playback does not match the user's requirements and the adjustment effect is poor.

In order for the regression model to accurately output the one or more target audio parameters, another example of the method for audio playback is provided.

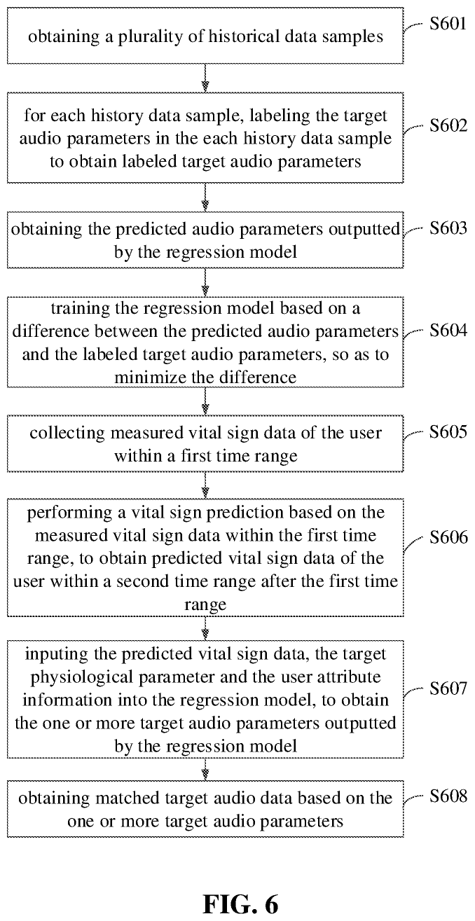

As illustrated in FIG. 6, the method for audio playback includes the following S601 to S608.

At S601, a plurality of historical data samples are obtained.

In this example, the historical data sample includes vital sign data of an individual, a target physiological parameter, user attribute information of the individual and one or more target audio parameters. The vital sign data includes measured vital sign data and predicted vital sign data. The vital sign data includes one or more selected from heart rate data, activity amount data, physiological pressure data, sleep data, emotion data and respiratory rate data. The user attribute information includes basic information of the user, the basic information of the user includes at least one of age, gender, height, weight or disease status.

In this example, the target audio parameters include target audio loudness and target rhythm.

In some implementations, the historical data sample is collected online. For example, data related to an individual wearing a wearable device, that is collected online by using web crawler technologies, is taken as the historical data sample.

In some implementations, the historical data sample is collected offline. For example, the data related to an individual wearing a wearable device, that is collected offline by participating in the test, is taken as the historical data sample, and the like.

At S602, for each history data sample, the target audio parameters in the history data sample are taken as labeled target audio parameters for label.

In the example, manual labeling is used to improve a training effect of the model.

In the example, for each history data sample including the predicted vital sign data, the target physiological parameters corresponding to user scenario information, the user attribute information and the target audio parameters, the target audio parameters including the target audio loudness and the target rhythm are used for label, and is taken as the labeled target audio parameters.

At S603, predicted audio parameters outputted by the regression model are obtained.

In the example, feature engineering can be firstly performed on the predicted vital sign data, the target physiological parameter and the user attribute information, so as to convert them into data that is suitable for the regression model to process.

Further, data processed by the feature engineering is inputted into the regression model, to obtain the target audio parameters outputted by the regression model.

At S604, the regression model is updated based on a difference between the predicted audio parameters and the labeled target audio parameters, so as to minimize the difference.

In the implementation of the present disclosure, after the predicted audio parameters outputted by the regression model are obtained, the predicted audio parameters are compared with the labeled target audio parameters to determine a difference between the predicted audio parameters and the labeled target audio parameters. Parameters of the regression model are adjusted according to the difference so as to minimize the difference, until a preset number of the update has been achieved or the difference has been reduced to meeting a preset condition, thereby obtaining a trained regression model that has learned the mapping relationship between the predicted vital sign data, the target physiological parameter and the user attribute information with the target audio parameters.

In some implementations, this training process is performed during the usage of the wearable device. For example, the vital sign data collected online in the previous audio playback process, and the loudness and rhythm of the audio selected by the user's operation are used as labels to train the regression model, thereby obtaining the trained regression model that has learned the mapping relationship between the predicted vital sign data, the target physiological parameter and the user attribute information with the target audio parameters. In this case, the vital sign data and the loudness and the rhythm of the audio selected by the user's operations are recorded in real time during the user's usage. The regression model is trained periodically or when a preset training condition is triggered, so as to obtain a regression model with a better adjustment effect.

In the example, the historical data samples are obtained from the server during the user's usage, and the historical data samples are used to train the regression model to obtain the regression model with a better adjustment effect.

At S605, measured vital sign data of the user within a first time range is collected.

At S606, a vital sign prediction is performed based on the measured vital sign data within the first time range, to obtain predicted vital sign data of the user within a second time range.

At S607, the predicted vital sign data, at least one target physiological parameter and user attribute information are inputted into the regression model, to obtain the one or more target audio parameters outputted by the regression model.

At S608, target audio data is obtained based on the one or more target audio parameters.

The execution of S101 to S104 can be referred to for the execution of S605 to S608, which will not be repeated herein.

Based on the above, a plurality of history data samples are obtained, and the target audio parameters in the history data sample are used to label the history data sample, and served as labeled target audio parameters. After the predicted audio parameters outputted by the regression model are obtained, the regression model is updated based on a difference between the predicted audio parameters and the labeled target audio parameters, so as to minimize the difference. Therefore, the regression model is trained, and the regression model has learned the mapping relationship between the predicted vital sign data, the target physiological parameter and the user attribute information with the target audio parameters, so that the regression model accurately outputs the target audio parameters.

An apparatus for audio playback is further provided in the disclosure.

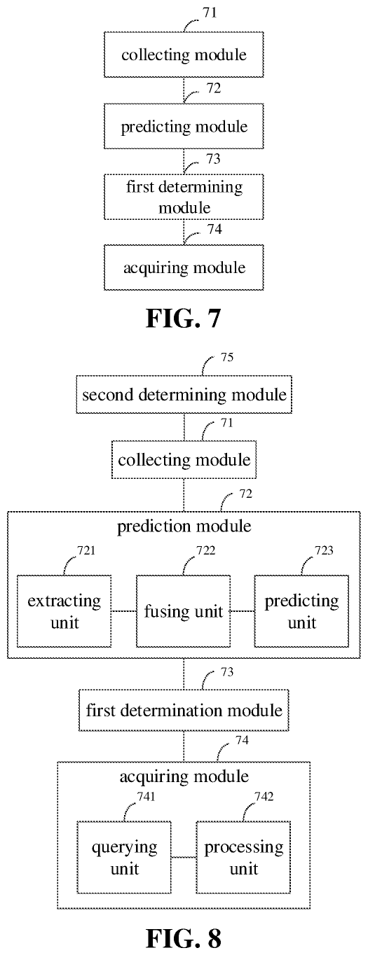

FIG. 7 is a structure diagram of an apparatus for audio playback provided in the implementation of the present disclosure.

As illustrated in FIG. 7, the apparatus for audio playback includes: a collecting module 71, a predicting module 72, a first determining module 73 and an acquiring module 74.

The collecting module 71 is configured to obtain measured vital sign data of a user within a first time range.

The predicting module 72 is configured to perform a vital sign prediction based on the measured vital sign data within the first time range, to obtain predicted vital sign data of the user within a second time range after the first time range.

The first determining module 73 is configured to determine one or more target audio parameters based on the predicted vital sign data and user attribute information of the user.

Specifically, the first determination module 73 is configured to input the predicted vital sign data, target physiological parameter and the user attribute information into a regression model, to obtain the one or more target audio parameters outputted by the regression model.

The one or more target audio parameters include target audio loudness and target rhythm. The user attribute information includes basic information of the user, the basic information of the user includes at least one of age, gender, height, weight or disease status. In some implementations, the user attribute information also includes environment information of the environment the user is in, and the environment information includes at least one of weather or time.

The acquiring module 74 is configured to obtain matched target audio data based on the one or more target audio parameters.

Further, the predicting module 72 includes an extracting unit 721, a fusing unit 722, and a predicting unit 723.

The extracting unit 721 is configured to perform feature extraction processing on the measured vital sign data within the first time range, to obtain first feature data.

The fusing unit 722 is configured to fuse the first feature data and the user attribute information, to obtain second feature data.

The predicting unit 723 is configured to perform prediction based on the second feature data, to obtain the predicted vital sign data within the second time range.

Furthermore, vital sign data of the user includes monitored data of heart rate and the monitored data of activity amount.

In some implementations, the extracting unit 721 is configured to: input the monitored data of the heart rate within the first time range into a shared layer of a prediction model for feature extraction, to obtain the first feature data corresponding to the heart rate; and input the monitored data of the activity amount within the first time range into the shared layer of the prediction model for feature extraction, to obtain the first feature data corresponding to the activity amount.

Furthermore, the predicted vital sign data includes prediction data of the heart rate and prediction data of the activity amount.

The predicting unit 723 is configured to: input the second feature data into a prediction layer of the prediction model corresponding to the heart rate to predict the heart rate, so as to obtain the prediction data of the heart rate; and input the second feature data into the prediction layer of the prediction model corresponding to the activity amount to predict the activity amount, so as to obtain the prediction data of the activity amount.

Specifically, the first determination module 73 is configured to input the predicted vital sign data, the target physiological parameter and the user attribute information into the regression model, to obtain the one or more target audio parameters outputted by the regression model. The one or more target audio parameters include the target audio loudness and the target rhythm.

In some implementations, the acquiring module 74 includes a querying unit 741 and a processing unit 742.

The querying unit 741 is configured to query matched audio elements on the server based on the target audio loudness and the target rhythm. Audio elements include a plurality of combinations of drum points, melodies, natural sounds and loudness.

The processing unit 742 is configured to synthesize queried audio elements to obtain target audio and play it.

In some implementations, the querying unit 741 is configured to send target audio modulation parameters to a server, so as to enable the server to query target audio data matching the target audio modulation parameters. The processing unit 742 is configured to obtain the target audio data sent by the server.

It needs to be noted that the foregoing explanation of the implementation of the method for audio playback is also applied to an apparatus for audio playback in the implementation, which will not be repeated herein.

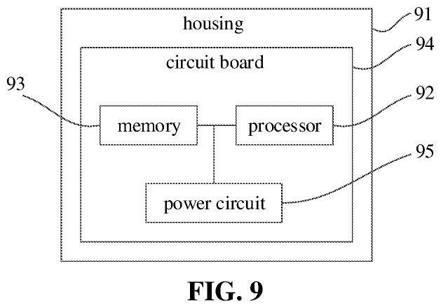

Based on the above implementations, the present disclosure further provides a possible implementation of an apparatus for audio playback. FIG. 8 is a structural diagram of an apparatus for audio playback provided in another implementation of the present disclosure. On the basis of the previous implementation, the apparatus for audio playback further includes a second determining module 75.

The second determining module 75 is configured to obtain historical heart rate data and historical activity amount data in the case that the scenario is set as a sleep aid scenario; determine historical activeness based on a weighted sum of the historical heart rate data and the historical activity amount data; determine the target physiological parameter corresponding to the sleep aid scenario, based on the activeness interval to which the historical activeness belongs.

In some implementations, the target physiological parameter includes the activeness. The second determining module 75 is configured to obtain historical heart rate data and historical activity data of the user within the third time range corresponding to the first time range; determine the historical activeness of the user based on the historical heart rate data and the historical activity data of the user; and determine the target activeness of the user according to the historical activeness of the user. The target activeness is lower than the historical activeness.

In the implementation of the present disclosure, measured vital sign data of a user within a first time range is obtained; a vital sign prediction is performed based on the measured vital sign data within the first time range, to obtain predicted vital sign data of the user within a second time range after the first time range; one or more target audio parameters are determined based on the predicted vital sign data and user attribute information of the user; and matched target audio data is obtained based on the one or more target audio parameters. Since the one or more target audio parameters are determined based on the predicted vital sign data, the target physiological parameter and the user attribute information, the target audio data matched with the one or more target audio parameters can be effective for adjusting sleep or health for the user, so as to solve the technical problem the related technologies that actual audio playback does not match requirements of the user and the adjustment effect is poor.

In order to achieve the above implementation, a wearable device is further provided in the disclosure. The wearable device includes at least one processor; and a memory communicatively connected to the at least one processor; the memory is stored with instructions executable by the at least one processor, and the instructions are performed by the at least one processor, so that the at least one processor performs the method for audio playback provided in the above any implementation of the disclosure.

FIG. 9 is a structural diagram of a wearable device provided in one implementation of the present disclosure, and flow of the implementations shown in FIGS. 1 to 8 in the present disclosure are realized.

As shown in FIG. 9, the wearable device includes a housing 91, a processor 92, a memory 93, a circuit board 94 and a power circuit 95. The circuit board 94 is disposed inside the space enclosed by the housing 91, and the processor 92 and memory 93 are disposed on the circuit board 94. The power circuit 95 is configured to supply power for each circuit or device of the wearable device. The memory 93 is configured to store executable program code. The processor 92 executes the program corresponding to the executable program code by reading the executable program code stored in the memory 93, and is used to execute the method for audio playback described in any of the aforementioned implementations.

The specific execution process of the above procedures by the processor 92 and the further execution procedures by the processor 92 by running executable program code are described in the implementations shown in FIGS. 1 to 8 of the present disclosure, which will not be repeated here.

In order to achieve the above implementation, a non-transitory computer readable storage medium stored with computer instructions is further provided, the computer instructions are configured to perform the method for audio playback as described in the above any implementation of the disclosure by a wearable device.

In the disclosure, descriptions with reference to terms “one implementation”, “some implementations”, “example”, “specific example” or “some examples” mean specific features, structures, materials or features described in combination with the implementation or example are included in at least one implementation or example of the disclosure. The schematic representations of the above terms do not have to be the same implementation or example. Moreover, specific features, structures, materials or features described are combined in one or more implementations or examples in a suitable manner. Furthermore, implementations or examples described in the specification, as well as features of implementations or examples, are combined without conflicting with each other.

In addition, the terms “first” and “second” are only for describing purposes and are not to be construed as indicating or implying relative importance or implicitly indicating the number of technical features indicated. Thus, features limiting “first” and “second” explicitly or implicitly include at least one of the features. In the description of the disclosure, “a plurality of” means at least two, for example, two, three, unless otherwise expressly and specifically stated.

Any process or method described in the flowchart or otherwise described herein is understood as representing one or more modules, segments, or portions of codes of executable instructions for implementing the blocks of a customized logical function or process, and the scope of the implementations of the present disclosure includes additional implementations, the functions are executed not in the sequence shown or discussed, including in a substantially simultaneous manner or in a reverse sequence, which will be appreciated by those skilled in the art the implementations of the disclosure belong to.

The logics and/or blocks represented in the flowchart or described in other ways herein, for example, are considered as an ordered list of executable instructions configured to implement logic functions, which are specifically implemented in any computer readable medium for use by a system, an apparatus or a device for executing instructions (such as a system based on a computer, a system including a processor, or other systems that obtain and perform instructions from a system, an apparatus or a device for performing instructions) or in combination with the system, the apparatus or the device for performing instructions. A “computer readable medium” in the disclosure is an apparatus that contains, stores, communicates, propagates or transmits a program for use by a system, an apparatus or a device for executing instructions or in combination with the system, the apparatus or the device for executing instructions. A more specific example (a non-exhaustive list) of a computer readable medium includes the followings: an electronic connector (an electronic apparatus) with one or more cables, a portable computer disk box (a magnetic device), a random access memory (RAM), a read-only memory (ROM), an electrically programmable read-only memory (an EPROM or a flash memory), an optical fiber apparatus, and a portable optical disk read-only memory (CDROM). In addition, a computer readable medium even is paper or other suitable medium on which a program is printed, since paper or other medium is optically scanned, and then edited, interpreted or processed in other suitable ways if necessary to obtain a program electronically and store it in a computer memory.

It should be understood that all parts of the present disclosure are implemented with a hardware, a software, a firmware and their combination. In the above implementation, a plurality of blocks or methods are stored in a memory and implemented by a software or a firmware executed by a suitable system for executing instructions. For example, if implemented with a hardware, they are implemented by any of the following technologies or their combinations known in the art as in another implementation: a discrete logic circuit with logic gate circuits configured to achieve logic functions on data signals, a special integrated circuit with appropriate combined logic gate circuits, a programmable gate array (PGA), a field programmable gate array (FPGA), etc.

Those skilled in the art understand that all or part of blocks in the above method implementations are implemented by instructing relevant hardware by computer programs. The programs are stored in a computer readable storage medium, and the programs include one of blocks of the method implementations or their combination when executed.

In addition, functional units in the implementations of the disclosure are integrated in one processing module, or each of the units is physically existed alone, or two or more units are integrated in one module. The integrated module is achieved by a form of a hardware, and also is achieved by a form of a software functional module. The integrated module is stored in a computer readable storage medium when it is implemented in a form of a software functional module and sold or used as an independent product.

The above storage medium is a read-only memory, a magnetic disk or an optical disk. Even though implementations of the disclosure have been illustrated and described above, it can be understood that the above implementations are exemplary and may not be construed as a limitation of the present disclosure, and changes, modifications, substitutions and alterations are made to the above implementations within the scope of the disclosure.

Those skilled in the art understand that all or part of processes in the above implementations are implemented by instructing relevant hardware by computer programs. The programs are stored in a computer readable storage medium, and the programs include processes of the above implementations when executed. The storage medium is a magnetic disk, an optical disk, a read-only memory (ROM) or a random access memory (RAM), etc.

The above are only specific implementations of the disclosure, however, the protection scope of the present disclosure is not limited here, and those skilled in the art easily think of any changes or substitutions within the technical scope of the present disclosure, which shall be within the protection scope of the disclosure. Therefore, the protection scope of the disclosure shall be subject to the protection scope of the claims.

What is claimed is:

1. A method for audio playback, comprising:
obtaining measured vital sign data of a user within a first time range;
performing a vital sign prediction based on the measured vital sign data within the first time range, to obtain predicted vital sign data of the user within a second time range after the first time range;
determining one or more target audio parameters based on the predicted vital sign data and user attribute information of the user; and
obtaining matched target audio data based on the one or more target audio parameters.

2. The method of claim 1, wherein obtaining measured vital sign data of the user within the first time range comprises:
obtaining the measured vital sign data of the user within the first time range based on user scenario information, wherein the user scenario information is used for indicating health requirements of the user.

3. The method of claim 2, wherein obtaining the measured vital sign data of the user within the first time range based on user scenario information comprises:
in response to the user scenario information indicating a sleep aid scenario or a focus scenario, obtaining heart rate data and activity data of the user within the first time range; or
in response to the user scenario information indicating a weight loss scenario, obtaining at least one of activity data, motion data, heart rate data, or positioning data of the user within the first time range; or
in response to the user scenario information indicating an emotion regulation scenario, obtaining heart rate variability of the user within the first time range.

4. The method of claim 1, wherein performing the vital sign prediction based on the measured vital sign data within the first time range, to obtain the predicted vital sign data of the user within the second time range after the first time range comprises:
performing feature extraction processing on the measured vital sign data within the first time range, to obtain first feature data;
performing fusing processing on the first feature data and the user attribute information, to obtain second feature data; and
performing the vital sign prediction based on the second feature data, to obtain the predicted vital sign data within the second time range.

5. The method of claim 1, wherein determining one or more target audio parameters based on the predicted vital sign data and user attribute information of the user comprises:
determining at least one target physiological parameter of the user; and
determining the one or more target audio parameters based on the predicted vital sign data, the at least one target physiological parameter and the user attribute information.

6. The method of claim 5, wherein determining the target physiological parameter of the user comprises:
determining the target physiological parameter of the user based on at least one of user scenario information or historical vital sign data of the user.

7. The method of claim 5, wherein determining the target physiological parameter of the user comprises:
in response to user scenario information indicating a sleep aid scenario or a focus scenario, determining a target activeness of the user; or
in response to the user scenario information indicating a weight loss scenario, determining at least one of the target activeness, a target activity amount or a target heart rate of the user; or
in response to the user scenario information indicating an emotion regulation scenario, determining a target heart rate variability of the user.

8. The method of claim 5, wherein, the target physiological parameter comprises a target activeness; and
determining the target physiological parameter of the user comprises:
obtaining historical heart rate data and historical activity data of the user within a third time range corresponding to the first time range;
determining a historical activeness of the user based on the historical heart rate data and the historical activity data of the user; and
determining a target activeness of the user based on the historical activeness of the user, wherein the target activeness is less than the historical activeness.

9. The method of claim 5, wherein determining one or more target audio parameters based on the predicted vital sign data and user attribute information of the user comprises:
processing the predicted vital sign data, the target physiological parameter and the user attribute information with a regression model, to obtain the one or more target audio parameters outputted by the regression model, wherein the one or more target audio parameters comprise a target audio loudness and a target rhythm.

10. The method of claim 1, wherein, the one or more target audio parameters comprise a target audio modulation parameter; and
obtaining matched target audio data based on the one or more target audio parameters comprises:
sending the target audio modulation parameter to a server, to enable the server to query target audio data matching the target audio modulation parameter; and
obtaining the target audio data sent by the server.

11. A method for audio playback, comprising:
determining a target physiological parameter of a user based on user scenario information, wherein the user scenario information is used for indicating health requirements of the user;
determining one or more target audio parameters based on the target physiological parameter of the user; and
obtaining matched target audio data based on the one or more target audio parameters.

12. The method of claim 11, wherein determining the target physiological parameter of the user based on the user scenario information comprises:
in response to the user scenario information indicating a sleep aid scenario or a focus scenario, determining a target activeness of the user; or
in response to the user scenario information indicating a weight loss scenario, determining at least one of a target activeness, a target activity amount and a target heart rate of the user; or
in response to the user scenario information indicating an emotion regulation scenario, determining a target heart rate variability of the user.

13. The method of claim 11, wherein determining the one or more target audio parameters based on the target physiological parameter of the user comprises:
obtaining vital sign data of the user based on the user scenario information; and
determining the one or more target audio parameters based on the target physiological parameter and the vital sign data of the user.

14. The method of claim 11, wherein determining the one or more target audio parameters based on the target physiological parameter of the user comprises:
obtaining measured vital sign data of the user within a first time range based on the user scenario information;
performing a vital sign prediction based on the measured vital sign data within the first time range, to obtain predicted vital sign data of the user within a second time range after the first time range; and
determining the one or more target audio parameters based on the predicted vital sign data and the target physiological parameter.

15. The method of claim 14, wherein performing the vital sign prediction based on the measured vital sign data within the first time range, to obtain the predicted vital sign data of the user within the second time range after the first time range comprises:
performing feature extraction processing on the measured vital sign data within the first time range, to obtain first feature data;
performing fusing processing on the first feature data and user attribute information, to obtain second feature data; and
performing the vital sign prediction based on the second feature data, to obtain the predicted vital sign data within the second time range.

16. The method of claim 14, wherein obtaining the measured vital sign data of the user within the first time range based on the user scenario information comprises:
in response to the user scenario information indicating a sleep aid scenario or a focus scenario, obtaining heart rate data and activity data of the user within the first time range; or
in response to the user scenario information indicating a weight loss scenario, obtaining at least one of activity data, motion data, heart rate data, or positioning data of the user within the first time range; or
in response to the user scenario information indicating an emotion regulation scenario, obtaining heart rate variability of the user within the first time range.

17. A wearable device, comprising:
at least one processor; and
a memory communicatively coupled to the at least one processor; wherein the memory is stored with instructions executable by the at least one processor, and execution of the instructions by the at least one processor causes the at least one processor to perform the method of claim 1.

18. A wearable device, comprising:
at least one processor; and
a memory communicatively coupled to the at least one processor; wherein the memory is stored with instructions executable by the at least one processor, and execution of the instructions by the at least one processor causes the at least one processor to perform the method of claim 11.

19. A non-transitory computer readable storage medium having stored therein computer executable instructions that, when executed by a processor, causes the processor to perform the method of claim 1.

20. A non-transitory computer readable storage medium having stored therein computer executable instructions that, when executed by a processor, causes the processor to perform the method of claim 11.
